(19) United States
(12) Patent Application Publication
(10) Pub. No.: US 2012/0289747 A1
(43) Pub. Date: Nov. 15, 2012

(54) CATALYSTS FOR THE HYDROGENATION OF AROMATIC AMINES

(75) Inventors: Lucia Königsmann, Stuttgart (DE); Thomas Heidemann, Viernheim (DE); Martin Bock, Neu-Ulm (DE); Joachim Pfeffinger, Ludwigshafen (DE); Benjamin Koch, Beimdersheim (DE)

(73) Assignee: Carl-Bosch-Strasse 38, Ludwigshafen (DE)

(21) Appl. No.: 13/468,689

(22) Filed: May 10, 2012

Related U.S. Application Data
(60) Provisional application No. 61/484,705, filed on May 11, 2011.

(51) Int. Cl. C07C 209/72; B01J 21/08

(52) U.S. Cl. 564/451; 502/261; 564/450

(57) ABSTRACT

The present invention relates to a catalyst comprising 4% by weight of ruthenium (Ru) or more and a support material comprising silicon dioxide, wherein the nitrogen content of the catalyst after the last drying or calcination is in the range from 1 to 3% by weight, and also catalyst precursors thereof. The present patent application further relates to a process for producing an Ru-comprising catalyst, which comprises the steps impregnation, drying, calcination and reduction. In addition, the present patent application relates to a process for hydrogenating organic substances in the presence of catalysts of the invention or catalysts produced according to the invention, and also a process for producing downstream products from cycloaliphatic amines prepared according to the invention.

The present application incorporates the preliminary U.S. Application No. 61/484,705, filed May 11, 2011, by reference.

The present invention relates to an Ru-comprising catalyst and also to a process for producing it, which comprises the steps impregnation, drying, calcination and reduction. In addition, the present patent application relates to a process for hydrogenating organic substances in the presence of catalysts of the invention or catalysts produced according to the invention, and also a process for producing downstream products from cycloaliphatic amines prepared according to the invention.

The hydrogenation of aromatic compounds is frequently carried out in the presence of catalysts comprising ruthenium as active metal.

Here, Ru can be used in the form of supported or unsupported catalysts.

DE-OS-2132547 describes a process for hydrogenating aromatic compounds to the corresponding cycloaliphatics. An unsupported catalyst based on oxide hydrates of Ru is used for the hydrogenation. The catalyst is produced by precipitation from an aqueous solution of an Ru salt by addition of alkali metal hydroxide. The Ru oxide hydrate obtained in this way can be used directly in the process or be subjected to drying before use. After drying, the catalyst is, according to the disclosure, present as powder having particle sizes in the range from 4 to 6 nm, with the Ru being present as Ru(IV) oxide hydrate comprising about 50% by weight of Ru in the dry powder obtained.

U.S. Pat. No. 3,864,361 describes the preparation of 2,5-dimethylpyrrolidone by reduction of 2,5-dimethyl-pyrrole in the presence of finely divided, unsupported RuO₂. The catalyst is removed by filtration after the hydrogenation is complete. According to the disclosure, the removal of the Ru catalyst can be improved by addition of Al₂O₃ as filter aid.

WO 2009/090179 discloses the hydrogenation of aromatic amines in the presence of unsupported Ru catalysts to which an inorganic additive is added during the reaction in order to reduce the tendency of the catalysts to agglomerate.

Supported Ru catalysts are disclosed, for example, in U.S. Pat. No. 5,981,801, EP 1366812, EP 0813906, EP 0814098 or DE 101 28 242.

U.S. Pat. No. 5,981,801 describes supported Ru catalysts on activated carbon, calcium carbonate, cerium dioxide, aluminum oxide, zirconium oxide, titanium dioxide or silicon dioxide. To increase the reaction rate and to reduce secondary reactions, the catalyst is pretreated with oxygen before use in the reaction.

EP 1366812 discloses a process for hydrogenating an aromatic amine, for example methylenedianiline (MDA), in the presence of a supported Ru catalyst. SiO₂ is mentioned as support material. The support material has, according to the invention, a BET surface area of from 30 m²/g to 70 m²/g.

EP 0813905 likewise describes the hydrogenation of aromatic amines in the presence of supported Ru catalysts. As support, mention is made of, inter alia, SiO₂. However, the BET surface area is not more than 30 m²/g.

The hydrogenation of aromatic amines is likewise disclosed in EP 0814098. As active metal, it is possible to use Ru either alone or together with other active metals. The support material, for example SiO₂, has a BET surface area of from 50 to 500 m²/g, preferably from 200 to 350 m²/g.

In DE 101 28 242, the hydrogenation of organic compounds is carried out in the presence of a catalyst comprising Ru as active metal, either alone or together with other active metals, with the active metals having been applied to a support material based on amorphous silicon dioxide. Ru is applied in the form of an aqueous solution of a halogen-free Ru compound, in particular Ru nitrosyl nitrate, to the support and the solid obtained in this way is subsequently dried at a temperature below 200° C. and then reduced.

For the purposes of the present invention, it has been found that the reduction of the catalysts described in DE 101 28 242 evolves, particularly at high Ru contents, a large quantity of heat which is undesirable from a safety point of view. In addition, the activity of the catalyst can be reduced by the evolution of heat.

It is an object of the present invention to provide improved catalysts for the hydrogenation of aromatic compounds. In particular, catalysts which have a high activity should be provided. A further object of the present invention is to provide calcined catalysts or catalyst precursors which evolve only a small quantity of heat during the reduction. This allows the reduction to be carried out more safely and makes it possible to produce Ru catalysts having a high Ru dispersity and also a high activity.

The object of the present invention has been achieved by a

process for producing a catalyst which comprises ruthenium and a support material comprising silicon dioxide and can be obtained by
a) single or multiple treatment of the support material with an aqueous solution of Ru nitrosyl nitrate and drying of the treated support material at a temperature below 250° C., subsequent
b) treatment of the support material resulting from step a) with an oxygen-comprising gas (calcination) in a temperature range from 100 to 250° C., and subsequent
c) reduction of the catalyst precursor obtained in step b) by means of hydrogen at a temperature in the range from 100 to 250° C.,
   and also by a catalyst which can be obtained by the abovementioned process.

The catalysts used in the process for hydrogenating aromatic compounds comprise Ru.

The catalyst can optionally comprise at least one further metal of transition group I, VII or VIII of the Periodic Table.

The catalyst preferably comprises Pd or Pt as further metal of transition group I, VII or VIII of the Periodic Table.

The molar ratio of Ru to the further metals of transition group I, VII or VIII of the Periodic Table is preferably from 100:0 to 100:20, particularly preferably from 100:0 to 100:10, very particularly preferably from 100:0.0001 to 100:5 and in particular from 100:0.001 to 100:1.

In a preferred embodiment of the present invention, ruthenium alone is used as active metal.

The catalysts used in the process for hydrogenating aromatic compounds further comprise a support material comprising silicon dioxide (SiO₂).

Support materials based on silicon dioxide are known to those skilled in the art and are commercially available (see, for example, O. W. Florke, “Silica” in Ullmann's Encyclopedia of Industrial Chemistry 5th ed. on CD-ROM). They can either be of natural origin or have been produced synthetically. Examples of support materials based on silicon dioxide are kieselguhr, silica gels, pyrogenic silica and precipitated silica.

In a preferred embodiment of the invention, the catalysts comprise silica gels as support materials.

The support material can optionally comprise further support materials such as Al₂O₃, MgO, CaO, TiO₂, ZrO₂, Fe₂O₃ or alkali metal oxide.

The proportion of SiO₂ in the support material is preferably in the range from 75 to 100% by weight, particularly preferably in the range from 90 to 100% by weight, very particularly preferably in the range from 95 to 99.9% by weight and in particular from 99 to 99.8% by weight, based on the support material used.

In a particularly preferred embodiment, SiO₂ is the sole support material.

In a further particularly preferred embodiment, pulverulent support material is used in the process of the invention for hydrogenating aromatic compounds.

The support material used preferably has an average particle size distribution (PSD) of from 0.1 to 1000 μm, particularly preferably from 1 to 500 μm and in particular from 2 to 200 μm. The average particle diameter d₅₀ is preferably in the range from 1 to 50 μm, particularly preferably in the range from 5 to 40 μm and very particularly preferably in the range from 10 to 30 μm.

The PSD is determined by means of laser light scattering in accordance with ISO 13320.

The support materials preferably have a mercury porosity (DIN 66133) in the range from 0.5 to 500 ml/g, particularly preferably from 1 to 300 ml/g and very particularly preferably from 1.5 to 200 ml/g. The average pore diameter is preferably in the range from 10 to 200 nm, particularly preferably in the range from 20 to 100 nm and very particularly preferably in the range from 25 to 50 nm.

The surface area of the support material is preferably from 50 to 500 m²/g, more preferably from 100 to 350 m²/g and in particular from 100 to 250 m²/g of the support.

The surface area of the support is determined by the BET method by means of N₂ adsorption, in particular in accordance with DIN 66131. The average pore diameter and the size distribution are determined by Hg porosimetry, in particular in accordance with DIN 66133.

In a further preferred embodiment, the support material can be used in the process of the invention in the form of shaped bodies which can be obtained, for example, by extrusion, ram extrusion or tableting and can, for example, have the shape of spheres, pellets, cylinders, rods, rings or hollow cylinders, stars and the like. The dimensions of these shaped bodies are preferably in the range from 1 mm to 25 mm. Greater preference is given to catalyst extrudates having extrudate diameters of from 2 to 5 mm and extrudate lengths of from 2 to 25 mm.

The catalysts used in the process of the invention can be obtained by
a) single or multiple treatment of the support material with an aqueous solution of Ru nitrosyl nitrate and drying of the treated support material at a temperature below 250° C., subsequent
b) treatment of the support material resulting from step a) with an oxygen-comprising gas (calcination) in a temperature range from 100 to 250° C., and subsequent
c) reduction of the catalyst precursor obtained in step b) by means of hydrogen at a temperature in the range from 100 to 250° C.

To produce the ruthenium catalysts used according to the invention, the support material is firstly treated with an aqueous solution of Ru nitrosyl nitrate in such a way that the desired amount of ruthenium is taken up by the support material. This step will hereinafter also be referred to as impregnation.

The treatment or impregnation of the support material can be carried out in various ways. For example, the support material can be sprayed or flushed with the Ru nitrosyl nitrate solution or the support matter can be suspended in the Ru nitrosyl nitrate solution. For example, the support material can be suspended in the aqueous solution of Ru nitrosyl nitrate and filtered off from the aqueous supernatant liquid after a particular time. The ruthenium content of the catalyst can then be controlled in a simple manner via the amount of liquid taken up and the ruthenium concentration of the solution. Impregnation of the support material can, for example, also be carried out by treating the support with a defined amount of the aqueous solution of Ru nitrosyl nitrate corresponding to the maximum amount of liquid which can be taken up by the support material. For this purpose, the support material can, for example, be sprayed with the amount of liquid. Suitable apparatuses for this purpose are those customarily used for mixing liquids and solids (see, for example, Vauck, Müller “Grundoperationen chemischer Verfahrenstechnik”, 10th edition, Deutscher Verlag für die Kunststoffundustrie, Leipzig, 1994, pp. 405ff.); particularly suitable apparatuses are tumble dryers, impregnation drums, drum mixers, paddle mixers and the like. Monolithic supports are usually flushed with the aqueous solutions of Ru nitrosyl nitrate.

For the present purposes, the term “aqueous” refers to water or mixtures of water with up to 50% by volume, preferably not more than 30% by volume and in particular not more than 10% by volume, of one or more water-miscible organic solvents, e.g. mixtures of water with C1-C4-alkanols such as methanol, ethanol, n-propanol or isopropanol. Water is frequently used as sole solvent. The aqueous solvent will frequently also comprise, for example, a halogen-free acid such as nitric acid, sulfuric acid or acetic acid to stabilize the Ru nitrosyl nitrate in the solution. The concentration of Ru nitrosyl nitrate in the aqueous solutions naturally depends on the amount of Ru nitrosyl nitrate to be applied and the uptake capacity of the support material for the aqueous solution and is generally in the range from 0.1 to 20% by weight.

After treatment of the support material with the aqueous Ru nitrosyl nitrate solution, the treated support material is generally separated off from the supernatant liquid and dried.

Drying is carried out in the temperature range below 250° C., particularly preferably below 200° C. and very particularly preferably below 150° C.

Drying is very particularly preferably carried out in the temperature range from 100 to 150° C. and in particular in the range from 110 to 130° C.

Drying of the support material which has been treated with Ru nitrosyl nitrate is usually carried out under atmospheric pressure, although reduced pressure can also be employed in order to promote drying. A gas stream, e.g. air or nitrogen, will frequently be passed over or through the material to be dried in order to promote drying.

The drying time naturally depends on the desired degree of drying and the drying temperature and is generally in the range from 2 hours to 30 hours, preferably in the range from 4 to 15 hours.

Drying of the treated support material is preferably continued until the content of water or of volatile solvent constituents before the reduction ii) is less than 5% by weight and in particular not more than 2% by weight, particularly preferably not more than 1% by weight, based on the total weight of the solid. The proportions by weight indicated are based on the loss in weight of the solid determined at a temperature of 300° C., a pressure of 1 bar and a time of 10 minutes.

Drying is preferably carried out with movement of the support material treated with the Ru nitrosyl nitrate solution, for example by drying the solid in a rotary tube oven or a rotary bulb oven. In this way, the activity of the catalysts of the invention can be increased further.

The treatment of the support material with an aqueous solution of Ru nitrosyl nitrate and the subsequent drying can be repeated when relatively large amounts of Ru are to be applied to the support material.

In the case of multistage impregnation processes, it is advantageous to carry out drying and optionally calcination between individual impregnation steps.

The catalyst support which has been treated according to the invention is brought into contact with an oxygen-comprising gas (calcination) after the last drying step.

The oxygen content of the oxygen-comprising gas is preferably from 0.1 to 25% by volume, particularly preferably from 0.5 to 21% by volume and very particularly preferably from 2 to 5% by volume. Particular preference is given to using air as oxygen-comprising gas. As an alternative, it is also possible to use mixtures of oxygen, inert gases (e.g. nitrogen or argon), hydrogen oxide (water vapor) and/or air.

The temperature in the calcination is in the range from 100 to 250° C., particularly preferably in the range from 150 to 250° C. and very particularly preferably in the range from 150 to 180° C.

The calcination time is preferably from 0.5 to 10 hours, particularly preferably from 1 to 5 hours and very particularly preferably from 2 to 4 hours.

The calcination can in each case be carried out batchwise, for example in a shaft oven, tray oven, muffle furnace, drying oven or in a fluidized-bed reactor, or continuously, for example in a rotary tube, belt calcination oven or rotary bulb oven.

The calcination generally gives a catalyst precursor in which Ru is at least partly present in the form of oxygen-comprising compounds, in particular as oxides, hydroxides and/or oxide hydrates.

In an embodiment of the present invention, 50 mol % or more, particularly preferably 75 mol % or more and very particularly preferably 90 mol % or more, of the ruthenium is present in the form of oxygen-comprising compounds after the calcination.

In a further embodiment, the Ru is essentially entirely present in the form of oxygen-comprising compounds after the calcination.

After the last drying or calcination step, the catalyst precursor preferably has a nitrogen content in the range from 1.0 to 3% by weight, preferably from 1.2 to 2.5% by weight and very particularly preferably from 1.3 to 2% by weight, based on the catalyst precursor.

The nitrogen content is determined in accordance with DIN 51732.

After the last drying or calcination step, the catalyst precursor has a water content of preferably less than 1% by weight, particularly preferably less than 0.1% by weight and in particular less than 0.01% by weight.

The conversion of the catalyst precursor into its catalytically active form is effected by reduction of the catalyst precursor.

For this purpose, the treated and calcined support material is brought into contact with hydrogen or a mixture of hydrogen and an inert gas.

The hydrogen partial pressure is preferably in the range from 0.2 bar to 1.5 bar.

The reduction of the catalyst precursor is preferably carried out at a hydrogen pressure of one atmosphere in a stream of hydrogen. The reduction is preferably carried out with movement of the catalyst precursor, for example by reduction of the catalyst precursor in a rotary tube oven or a rotary bulb oven. In this way, the activity of the catalysts of the invention can be increased further.

Reduction is carried out at a temperature in the range from 100 to 250° C., preferably in the range from 150 to 230° C. and particularly preferably in the range from 180 to 220° C.

The ruthenium content of the reduced catalyst is preferably 4% by weight or more, particularly preferably 6% by weight or more, very particularly preferably 7% by weight or more and in particular 8% by weight or more.

In a particular embodiment, the Ru content is preferably in the range from 4 to 30% by weight, particularly preferably from 4 to 20% by weight, very particularly preferably from 6 to 15% by weight and in particular from 7 to 12% by weight, based on the total weight of the reduced catalyst.

Preference is given to 95 mol % or more, particularly preferably 98 mol % or more, very particularly preferably 99 mol % or more, of the ruthenium atoms in the reduced catalyst having an oxidation number of 0. In a particularly preferred embodiment, essentially all ruthenium atoms have the oxidation number 0.

The catalyst of the invention preferably has an Ru dispersity (measured in accordance with DIN 66136) in the range from 5 to 50%, particularly preferably from 10 to 30% and very particularly preferably from 15 to 25%.

After the reduction, the catalyst obtained in this way can be passivated to improve handling, e.g. by briefly treating the catalyst with an oxygen-comprising gas, e.g. air, but preferably with an inert gas mixture comprising from 1 to 10% by volume of oxygen.

Aromatic compounds which can be hydrogenated to cycloaliphatic amines are used in the process of the invention.

Aromatic compounds which can be hydrogenated to cycloaliphatic amines are usually monocyclic or polycyclic aromatic compounds which comprise one or more nitrogen-comprising substituents.

In a preferred embodiment, aromatic compounds which have one or more nitrogen-comprising substituents and in which the nitrogen atom of the nitrogen-comprising substituents is bound directly to the aromatic ring (N-substituted aromatic compounds) are used.

Preference is given to using aromatic monoamines, diamines or polyamines which can be hydrogenated to the corresponding cycloaliphatic amines.

Possible aromatic amines are monocyclic or polycyclic aromatic compounds having one or more amine groups, for example:

aromatic monoamines such as aniline, the isomeric toluidines, the isomeric xylidines, 1- or 2-aminonaphthalene, benzidine and substituted benzidines;
aromatic diamines such as the isomeric phenylenediamines, the isomeric toluenediamines, the isomeric diaminonaphthalenes, 4,4′-diamino-3,3′-dimethyldiphenylmethane, 4,4′-diamino-3,3′,5,5′-tetramethyldiphenylmethane and 4,4′-diaminodiphenylmethane; or
aromatic polyamines such as polymeric MDA (polymethylene-polyphenyl-amine).

As aromatic compounds which can be hydrogenated to cycloaliphatic amines, it is also possible to use aromatics having nitro, nitrile and urethane groups as substituents on the aromatic ring, for example

aromatic compounds having nitro groups as substituents, e.g. nitrobenzene, nitrotoluene, dinitrobenzene, dinitrotoluene and the isomeric nitroanilines;
aromatic compounds having nitrile groups as substituents, e.g. benzonitrile, tolunitrile or o-aminobenzonitrile; or
aromatic compounds having urethane groups as substituents, for example the dialkylurethanes which are formed from 4,4′-methylenedi(phenyl diisocyanate), 2,4′-methylenedi(phenyl diisocyanate) or 2,2′-methylenedi(phenyl diisocyanate) and aliphatic alcohols such as C₁-C₆-alcohols, in particular n-butanol,
the dialkylurethanes which are formed from tolylene 2,4-diisocyanate or tolylene 2,6-diisocyanate and aliphatic alcohols such as C₁-C₆-alcohols, in particular n-butanol, the dialkylurethanes which are formed from polymeric diphenylmethane diisocyanate and aliphatic alcohols such as C₁-C₆-alcohols, in particular n-butanol,
the dialkylurethanes which are formed from phenylene 2,4-diisocyanate or phenylene 2,6-diisocyanate and aliphatic alcohols, such as C₁-C₆-alkohols, in particular n-butanol, or the dialkylurethanes which are formed from naphthylene 1,5-diisocyanate and aliphatic alcohols such as C₁-C₆-alcohols, in particular n-butanol.

In addition to the substituents which can be hydrogenated to form amine groups, the aromatic compounds can have no further substituents or they can bear one or more further substituents, for example alkyl, cycloalkyl, aryl, heteroaryl, halo, haloalkyl, silyl, hydroxy, alkoxy, aryloxy, carboxy or alkoxycarbonyl substituents.

Preference is given to using aromatic amines such as the abovementioned aromatic monoamines, diamines and/or polyamines in the process.

Particular preference is given to using polymeric MDA, aniline, 2,4-diaminotoluene, 2,6-diaminotoluene, o-phenylenediamine, m-phenylenediamine, p-phenylenediamine, 4,4′-diamino-3,3′-dimethyldiphenylmethane, 4,4′-diamino-3,3′,5,5′-tetramethyldiphenylmethane and/or 4,4′-diaminodiphenylmethane in the process.

Very particular preference is given to using aniline, 4,4′-diamino-3,3′-dimethyldiphenylmethane, 4,4′-diamino-3,3′,5,5′-tetramethyldiphenylmethane and/or 4,4′-diaminodiphenylmethane.

In a particular embodiment, aromatic compounds which can be hydrogenated to the corresponding cycloaliphatic amines and comprise by-products are used in the process of the invention. Examples of such by-products are hydrochlorides of the aromatic starting amines or higher-boiling aromatic by-products. The term higher-boiling by-products refers to constituents which have a boiling point higher than that of the aromatic starting compounds which are to be hydrogenated to the cycloaliphatic products.

The chlorine content of the aromatic compounds which can be hydrogenated to the corresponding cycloaliphatic amines is preferably 1 ppm or more, more preferably from 10 ppm to 10 000 ppm and particularly preferably from 20 ppm to 1000 ppm, with the chlorine content usually being determined in accordance with DIN V 51408 part 2.

The content of higher-boiling aromatic compounds in the starting compounds is generally 1% by weight or more, preferably from 2 to 20% by weight and particularly preferably from 2 to 10% by weight, with the content of higher-boiling aromatic compounds being determined by laboratory distillation in accordance with ASTM D 5236-03 at a pressure of 1 mbar and a temperature up to 260° C.

A hydrogen-comprising gas is used in the process of the invention.

The hydrogen is generally used as technical-grade hydrogen. The hydrogen can also be used in the form of a hydrogen-comprising gas, i.e. in mixtures with other inert gases such as nitrogen, helium, neon, argon or carbon dioxide. As hydrogen-comprising gases, it is possible to use, for example, reformer offgases, refinery gases, etc., if and insofar as these gases do not comprise any catalyst poisons for the Ru-comprising catalysts, for example CO. However, preference is given to using pure hydrogen or essentially pure hydrogen in the process.

The preparation of the aromatic amines is carried out in the presence of the above-described Ru-comprising catalysts of the invention.

The hydrogenation can be carried out batchwise or continuously.

In a batch reaction, the hydrogenation can, for example, be carried out in a stirred vessel or stirring autoclave, a loop reactor, a jet loop reactor, a bubble column or a reactor having pump circulation. The batch hydrogenation is preferably carried out in a stirred vessel or stirring autoclave.

In a continuous reaction, the hydrogenation is usually carried out in a continuously operated stirred tank reactor, a continuously operated loop reactor, a continuously operated jet loop reactor, a continuously operated bubble column or a continuously operated reactor having pump circulation or a cascade of stirred vessels.

The process of the invention is generally carried out at a pressure of 50-350 bar, with preference being given to employing a pressure of from 150 to 250 bar.

As a rule, the process is carried out at a temperature in the range from 30 to 280° C., with the temperature range from 120 to 260° C. being particularly preferred.

The hydrogenation can be carried out with or without solvent. Solvents used are alcohols such as isopropanol, isobutanol or t-butanol or ethers such as diethyl ether, glycol dimethyl ether, dioxane or tetrahydrofuran.

However, the end product formed in the reaction can also be used as solvent.

Mixtures of the abovementioned solvents are also possible as solvents.

Preferred solvents are isopropanol, isobutanol and/or t-butanol. Particular preference is given to using the end product formed in the reaction as solvent.

The solvent is usually employed in such an amount that from 10 to 50% strength (% by weight), preferably from 15 to 40% strength, particularly preferably from 20 to 30% strength, solutions of the aromatic compounds provided for hydrogenation are obtained.

When the process is carried out continuously, it is particularly advantageous to employ the end product formed in the reaction as solvent.

The Ru-comprising catalyst is preferably used as a suspension of the catalyst in the liquid starting materials or solvents used.

When the process is carried out batchwise, the Ru catalyst is usually introduced, either as dry powder or as filtercake moist with water, directly into the hydrogenation reactors.

The Ru catalyst is particularly advantageously mixed with a solvent, the liquid starting material or the liquid reaction output to give a suspension which can then be fed into the reactor by means of suitable metering pumps. In a continuous mode of operation, this catalyst suspension is usually fed continuously into the hydrogenation reactor.

The reaction mixture from the hydrogenation is usually purified.

Purification of the reaction mixture is usually carried out by rectification or distillation.

The inorganic additive and the heterogeneous Ru-comprising catalyst can be removed, for example by solid-liquid separation such as filtration, sedimentation or centrifugation, before the distillation.

Solvents and unreacted starting materials can be recirculated to the process.

The cycloaliphatic amines which can be obtained by the process of the invention can be used as synthetic building blocks for the production of surfactants, drugs and crop protection agents, stabilizers, light stabilizers, polymers, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators, emulsifiers and/or as starting substances for the preparation of ureas and polyureas.

In particular, cyclohexylamine which can be obtained by the hydrogenation of aniline can be used as corrosion inhibitor or vulcanization accelerator.

4,4′-Diaminodicyclohexylmethane, 4,4′-diamino-3,3′,5,5′-tetramethyldicyclohexylmethane, 2,4-diaminomethylcyclohexane, 2,6-diaminomethylcyclohexane or 4,4′-diamino-3,3′-dimethyl-dicyclohexylmethane can be used as monomer building blocks for polyamides, as hardeners for epoxy resins or as starting material for the preparation of the corresponding isocyanates.

The present invention thus also provides a process for producing surfactants, drugs and crop protection agents, stabilizers, light stabilizers, polymers, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators, emulsifiers and/or as starting substances for the preparation of ureas and polyureas, wherein cycloaliphatic amines are prepared in a first stage by a process according to claim 8 and the cycloaliphatic amines obtained in the first stage are used for producing surfactants, drugs and crop protection agents, stabilizers, light stabilizers, polymers, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators, emulsifiers and/or as starting substances for the preparation of ureas and polyureas.

The present invention makes it possible to provide catalysts which are suitable for hydrogenating aromatic compounds and have a high activity. The reduction of the catalyst precursors according to the invention to form the actual catalyst evolves only little heat. This makes it possible for the reduction of the catalyst precursor to be carried out safely. In particular, the catalyst precursors of the invention have a high Ru dispersity and make it possible to produce catalysts having a high activity.

Further advantages of the process of the invention are that the process of the invention generally gives a high space-time yield. In addition, the process usually ensures a stable operation.

The supported catalysts can be separated from the reaction mixture and be reused in the process.

The present invention is illustrated by the following examples.

### EXAMPLES

### Catalyst Production

### Catalyst 1.1

### Comparative Example

107.87 g of Ru nitrosyl nitrate solution were diluted with distilled H₂O to a solution volume of 116 ml in a measuring cylinder. 100 g of support (Sipernat D120 from Evonik) were placed in an impregnation drum and the impregnation solution was subsequently divided into four and added to the support. After impregnation, the solid was dried at 120° C. for 16 hours. After drying, the catalyst comprised 3.2% of N.

DSC measurements (dynamic differential calorimetry, DIN 51007) under H₂ showed that the onset temperature was 163° C. and the heat liberated was 1350 J/g.

Drying was followed directly by reduction using H₂ and N₂ in a rotary tube oven at 200° C. for a period of 2 hours. The heating-up time was 20 minutes. After the reduction, the catalyst was cooled under nitrogen and passivated by means of a 5% nitrogen-air mixture.

### Catalyst 1.2

### According to the Invention

Impregnation process and drying were carried out in a manner analogous to method 1.1.

After drying, the catalyst was calcined at 180° C. for 3 hours. Elemental analysis of the calcined catalyst precursor gave an N content of 1.5%.

After the calcination, the catalyst precursor was reduced and passivated.

DSC measurements (dynamic differential calorimetry) under H₂ after calcination showed that the onset temperature was 420° C. and the heat liberated was 50-100 J/g.

### Catalyst 1.3

### Comparative Example

Impregnation process and drying were carried out in a manner analogous to method 1.1. After drying, the catalyst was calcined at 300° C. for 3 hours (elemental analysis: N content<0.5%). The calcined catalyst precursor was subsequently reduced and passivated.

### Catalyst 1.4

### According to the Invention

Impregnation process and drying were carried out in a manner analogous to method 1.1. After drying, the catalyst was calcined at 180° C. for 3 hours (elemental analysis: N content=1.5%). The catalyst precursor was used in the reaction without reduction.

### Properties of Catalysts 1.1-1.4:

The properties of the catalysts are shown in table 1. The Ru dispersity, the Ru surface area, the pore volume, the BET surface area, the Ru content and the Ru crystallite size were measured after reduction. Only the N content was determined before reduction, after the last drying and calcination steps.

TABLE 1

| | Catalyst 1.1 | | Catalyst 1.3 | |
|---|---|---|---|---|
| | (comparative | Catalyst | (comparative | Catalyst |
| | cat.) | 1.2 | cat.) | 1.4 |
| Ru dispersity (%) | 22.3 | 17.2 | 4.7 | 13.3 |
| Ru surface area | 6.8 | 5.9 | 1.6 | 4.3 |
| (m²/g of sample) | | | | |
| Pore volume (ml/g) | 2.6 | 2.6 | 2.1 | 2.5 |
| BET (m²/g) | 108 | 107 | 114 | 108 |
| N content after | 3.2 | 1.5 | <0.5 | 1.5 |
| calcination (%) | | | | |
| DSC (energy liberated | 1350 | 100 | <100 | 100 |
| in J/g) | | | | |
| Ru crystallite | ≦4 | ≦5 | 18.5 | <5 |
| size from XRD | | | | |
| (Ru in nm) | | | | |
| Ru content | 9.5 | 9.4 | 9.2 | 8.6 |
| (% by weight) | | | | |

### 2. Hydrogenation

### 2.1. Hydrogenation of O-Toluidine Base

### 2.1.1 Comparison of Various Catalysts

226 mg of the catalyst produced according to example 1.1 were placed in a 500 ml pressure reactor and admixed with 181 g of a 17.7% strength solution of o-toluidine base in THF. The hydrogenation was carried out using pure hydrogen at a constant pressure of 75 bar and a temperature of 180° C. for a period of 2.5 hours. The reactor was subsequently depressurized. The conversion was 100% at a selectivity of 98.15% (see table 1).

The catalysts 1.2-1.4 were used in a manner analogous to the method 2.1.1. The results are likewise shown in table 2.

TABLE 2

Time/min

| | | 30 | 120 | 180 | 300 |
|---|---|---|---|---|---|
| Catalyst 1.1 | DMDC | 19.88 | 85.23 | 98.15 | |
| (comparative | H6-oTB | 66.67 | 12.94 | 0 | |
| example) | oTB | 12.66 | 0 | 0 | |
| Catalyst 1.2 | DMDC | 14.15 | 93.7 | 98.37 | |
| (according to | H6-oTB | 65.59 | 4.73 | 0 | |
| the invention) | oTB | 19.56 | 0 | 0 | |
| Catalyst 1.3 | DMDC | 2.63 | 13.98 | 28.06 | 48.69 |
| (comparative | H6-oTB | 34.78 | 64.42 | 64.35 | 48.32 |
| example) | oTB | 61.6 | 20.03 | 5.83 | 0.8 |
| Catalyst 1.4 | DMDC | 16.36 | 88.63 | 98.01 | |
| (according to | H6-oTB | 64.23 | 9.37 | 0 | |
| the invention) | oTB | 17.222 | 0 | 0 | |

DMDC: 2,2′-dimethyl-4,4′-methylenedicyclohexylamine
H6-oTB: semihydrogenated o-toluidine base
oTB: o-toluidine base

### 2.2.2. Comparative Example According to DE10128242 (3% of Ru/SiO₂, Catalyst A)

The catalyst was produced in a manner analogous to the method described in DE10128242 for “catalyst A”. After drying, the N₂ content was 0.9%.

The catalyst was operated in a manner analogous to experiment 2.1.1.

The same concentration (in ppm by weight) of Ru based on the o-toluidine base used was used.

The results are likewise shown in table 3.

TABLE 3

| | 3% of Ru/SiO₂ according to DE10128242 | | |
|---|---|---|---|
| Time/min | DMDC | H6-oTB | oTB |
| 30 | 2.71 | 29.52 | 66.64 |
| 120 | 27.19 | 62.08 | 7.81 |
| 180 | 61.5 | 34.34 | 0.46 |

1.-14. (canceled)

15. A process for producing a catalyst which comprises ruthenium and a support material comprising silicon dioxide,
the process comprising:
a) single or multiple treatment of the support material with an aqueous solution of Ru nitrosyl nitrate and drying of the treated support material at a temperature below 250° C.,
b) treatment of the support material resulting from step a) with an oxygen-comprising gas (calcination) in a temperature range from 100 to 250° C. to obtain a catalyst precursor, and
c) reduction of the catalyst precursor obtained in step b) by means of hydrogen at a temperature in the range from 100 to 250° C.

16. The process for producing a catalyst according to claim 15, wherein the ruthenium content of the catalyst is in the range from 4 to 30% by weight, based on the total mass of the catalyst.

17. The process for producing a catalyst according to claim 15, wherein the Ru dispersity in accordance with DIN 66136 is in the range from 5 to 50%.

18. A process for producing a catalyst according to claim 15, wherein the support material has a mercury porosity according to DIN 66133 in the range from 0.5 to 500 ml/g.

19. A process for producing a catalyst according to claim 15, wherein the support material has a surface area in the range from 50 to about 500 m²/g.

20. A process for producing a catalyst according to claim 15, wherein the support material has an average particle size distribution of from 0.1 to 1000 μm.

21. A process for producing a catalyst according to claim 15, wherein the ruthenium content of the catalyst is from 4 to 30% by weight.

22. A process for producing a catalyst according to claim 15, wherein 95 mol % or more of the ruthenium atoms in the catalyst have an oxidation number of 0.

23. A catalyst obtained by the process according to claim 15.

24. A process for hydrogenating at least one organic compound by bringing the at least one organic compound into contact with a hydrogen-comprising gas in the presence of a catalyst, which has been produced by the process according to claim 15.

25. The process according to claim 24 for hydrogenating aromatic amines to cycloaliphatic amines.

26. The process according to claim 25 for hydrogenating polymeric MDA, aniline, 2,4-diaminotoluene, 2,6-diaminotoluene, o-phenylenediamine, m-phenylenediamine, p-diphenylenediamine, 4,4′-diamino-3,3′-dimethyldiphenylmethane, 4,4′-diamino-3,3′,5,5′-tetramethyldiphenylmethane and/or 4,4′-diaminodiphenylmethane.

27. A process for producing surfactants, drugs and crop protection agents, stabilizers, light stabilizers, polymers, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators, emulsifiers and/or as starting substances for the preparation of ureas and polyureas, wherein cycloaliphatic amines are prepared in a first stage by the process according to claim 24 and the cycloaliphatic amines obtained in the first stage are used for producing surfactants, drugs and crop protection agents, stabilizers, light stabilizers, polymers, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators, emulsifiers and/or as starting substances for the preparation of ureas and polyureas.

28. A process for producing cycloaliphatic amines comprising utilizing the catalyst according to claim 23.
